(19) United States
(12) Patent Application Publication
(10) Pub. No.: US 2024/0024672 A1
(43) Pub. Date: Jan. 25, 2024

(54) System for Pain Relief With TENS unit, garment, and Optional Heat

(71) Applicant: Cynthia Scribner, York, SC (US)

(72) Inventor: Cynthia Scribner, York, SC (US)

(21) Appl. No.: 18/225,001

(22) Filed: Jul. 21, 2023

Related U.S. Application Data
(60) Provisional application No. 63/391,214, filed on Jul. 21, 2022.

(51) Int. Cl. A61N 1/36; A61N 1/04

(52) U.S. Cl. CPC A61N 1/36021; A61N 1/0456; A61N 1/0484

(57) ABSTRACT

A system for providing pain relief and heat for those with poor circulation or in cold environments. The system includes a garment with inner pockets of a suitable size and shape to hold TENS unit electrodes in place during use. The material between the electrodes and the user is conductive, so adhesive and conductive gel are not required to convey the electrical stimulation from the electrode to the user. The TENS unit may also be used to power heating for the user.

### CROSS-REFERENCE TO RELATED APPLICATIONS

This application claims priority to provisional application No. 63/391,214.

### BACKGROUND OF THE INVENTION

A known pain relief device, called a TENS (Transcutaneous Electrical Nerve Stimulation) unit, is typically used by attaching electrodes to the patient with adhesive in a pattern around the area or areas in pain. This is time consuming, delaying pain relief, and the user must later wash the adhesive off, which is further complicated by the temporary nature of the pain relief. People with mobility issues may not even be capable of applying the electrodes to themselves without assistance, which increases time and at least embarrassment, and often also expense.

People with diseases causing poor circulation, (ex: Neuropathy, Diabetes, congestive heart failure) suffer greatly with ice cold feet that can lead to anything from minor discomfort through major discomfort to possible amputation. Regular socks, even the so-called warm ones, do not generate heat, and because the patient is not able to generate heat the socks can remain cold without anyone knowing, including themselves, that their feet aren't warm enough for safety.

### SUMMARY OF THE INVENTION

A system for relieving pain, which includes a TENS unit having a power source and regulator, wires, electrodes, and a garment with inner pockets which are sized to hold the electrodes in place. The pockets are constructed to permit electrical contact between the electrodes and the wearer while the electrodes are in the pockets.

They system also includes optional heating, for those who find heat also helps with pain relief, with the heating done either with electrical components also powered by the TENS unit or by chemical or thermal storage.

The disclosed system is intended for use with a wide variety of health conditions. Any condition for which a TENS unit is a known pain treatment method is suitable for treatment with the inventive system.

### BRIEF DESCRIPTION OF DRAWINGS

FIG. 1: the system, executed as a top, without heating components.

FIG. 2: the electrical parts of the system, not inserted into the garment, without heating components

FIG. 3: the electrical parts of the system, not inserted into the garment, with electrical heating components

FIG. 4: the system, executed as pants, with heating components

FIG. 5: a typical TENS unit, PRIOR ART

### DETAILED DESCRIPTION OF THE INVENTION

In the following description, numerous specific details are set forth to clearly describe the embodiments disclosed herein. However, one skilled in the art will understand that some well known features have not been described in detail so as not to obscure the invention.

In the following description, like reference numbers are used to identify like elements. Furthermore, the drawings are intended to illustrate major features of the exemplary embodiments in a diagrammatic manner. The drawings are not intended to depict every feature of every implementation and are not drawn to scale.

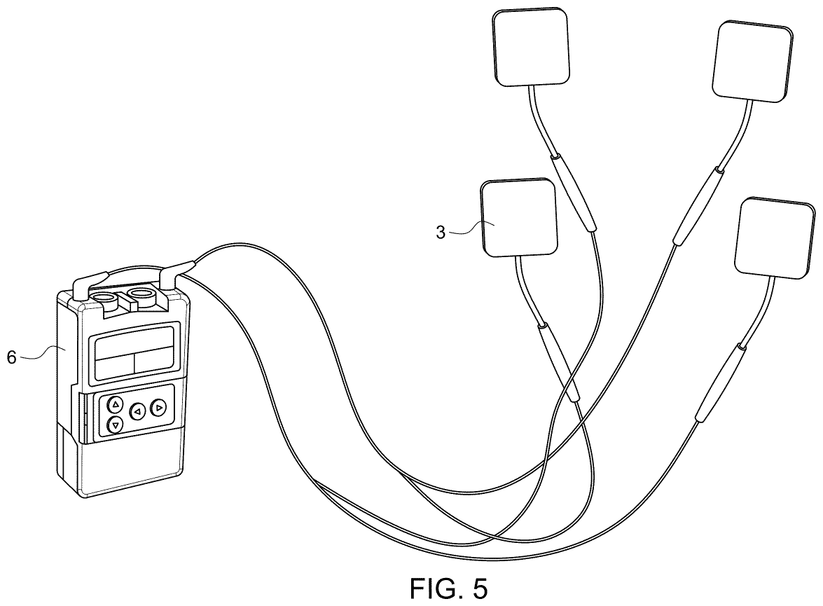

As illustrated in FIG. 5, a TENS unit typically includes a power source with frequency and intensity control built into the power source (4), wires (5), and electrodes (3) connected to the user via adhesive and conductive gel.

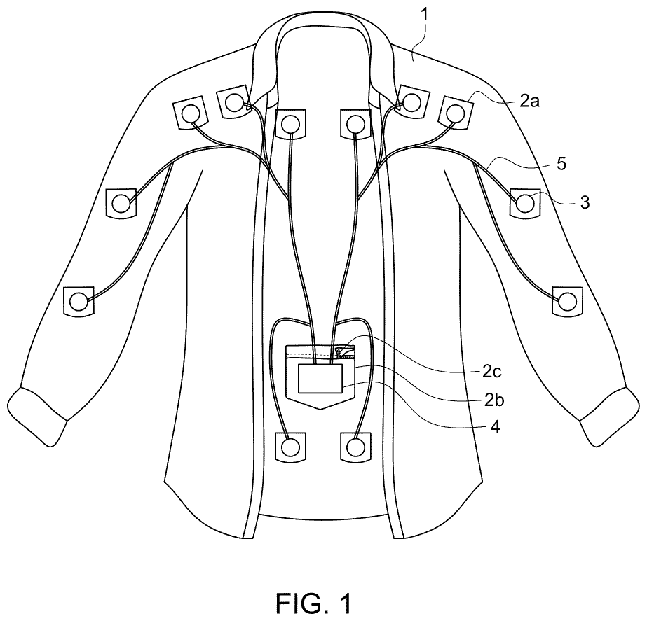

As illustrated in FIG. 1, the system includes a base garment (1), inner pockets (2), electrodes within the inner pockets (3), a TENS unit power source (4), and wires (5) connecting the TENS unit power source to the electrodes. There is an additional inner pocket (2b) into which the TENS unit power source is placed while in use. All of the inner pockets (2a, 2b) may be closed with hook and loop fasteners (2c), commonly known as Velcro, permitting a secure closure despite wires passing through.

The side of the inner pocket in contact with the wearer is constructed to permit electrical contact between the TENS unit electrode and the wearer. This may be achieved by the use of conductive material for part or all of the pocket facing the wearer. The entire base garment, including the pockets, is machine washable and dryer safe for ease of cleaning. The electrical contact may also be achieved by sewing small filament wiring or conductive thread through the inner side of the inner pocket. The material used may be stretch conductive fabric, non-stretch conductive fabric, or any other suitably washable and conductive material.

Although the details of the structure of the electrodes are not shown, the electrodes are preferably made to be sufficiently sturdy that they are not disposable, but instead are simply removed for washing and then used again indefinitely.

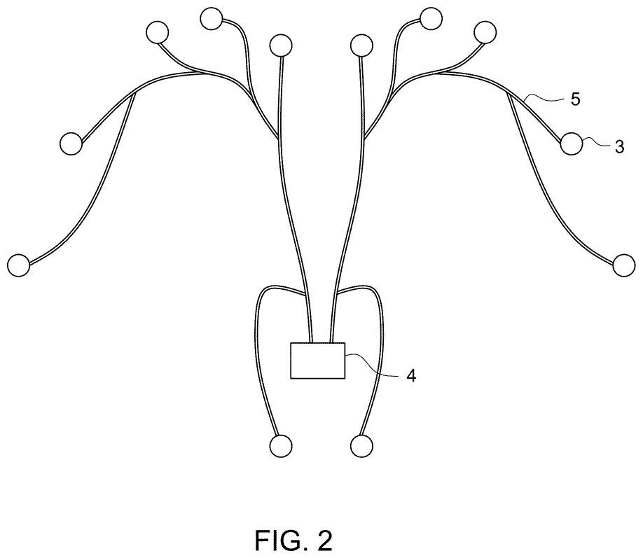

FIG. 2 illustrates the components of the system which as removed when washing the base garment. These include the TENS unit power source (4), wires (5), and electrodes (3).

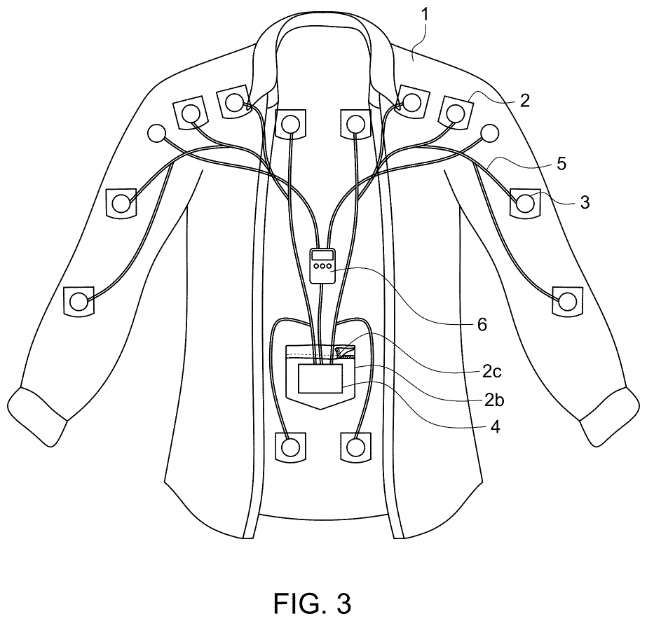

FIG. 3 illustrates the components of the system with heat as removed for washing the base garment. These include the TENS unit power source (4), wires (5), electrodes (3), and heating wires (6). The heating wires (6) are similar to those used in conventional electrically powered heating blankets or garments, but are intentionally easily removed so the garment can be washed. The heating wires and tens unit wires are both held in place via conventional means, ideally Velcro for ease of placement and removal. All wires (5, 6) have suitable connectors for attachment to the TENS unit power source (4), which is conventional, and the wires used for the electrodes have suitable connectors for attachment to the electrodes.

Known TENS unit power sources include an electronic pulse generator like, for example, Heatlhmateforever® unit model YK15AB or another Transcutaneous Electrical Nerve Stimulation unit, and a power source like, for example, NanoBolt lithtungten (zinc, oxide, manganese) battery, FLCB (Flexible Lithium Ceramic Battery generally made with copper, nickel, and tin) or PLCB (Pouch Lithium Ceramic battery, generally made with copper and beryllium), or any other known power source which performs comparably and safely.

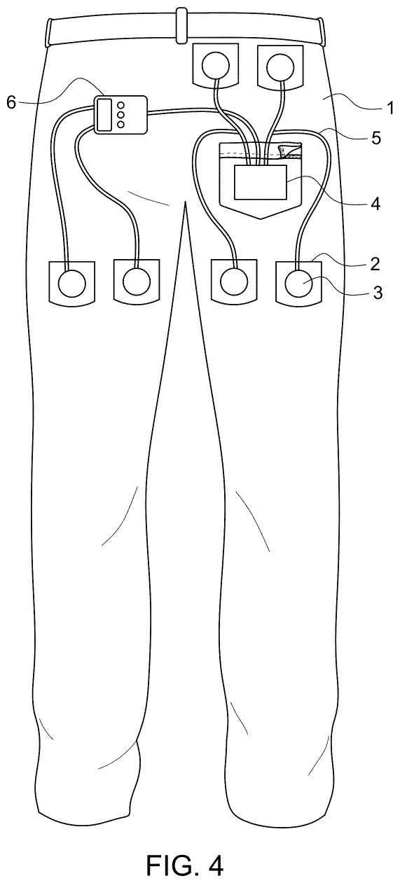

FIG. 4 illustrates the system in an embodiment with heating, executed as pants instead of a top.

FIG. 5 illustrates a typical TENS unit, including conventional electrode pads, which have a conductive gel and an adhesive. The parts of a TENS unit electrode that wear out are the gel and adhesive; the metal electrode itself generally does not.

In the most comprehensive embodiment, the entire system includes both a top and pants, with the pockets for electrodes being located above and below each knee in front, in the outer pelvic area of each hip, one on each pectoralis major between armpit and collarbone, 2 spaced over each shoulder, 2 each across the forearms, one on top of each foot, on the outer side above each ankle, one at the back of each heel, 1 each above and below the back of each knee, 1 each on the hips above the buttocks; one on each side of the lower back, one on each side of the back between the spine and shoulder blades, and one each for the tops of the hands.

Because different users have different locations in which a TENS unit may be helpful for pain reduction, it is anticipated that many users will not need at least some of the electrodes. For example, someone addressing knee pain alone will only need to have electrodes positioned to mitigate knee pain, above and below the front and back of the knee or knees in question. This can be done by having a garment which only has pockets for electrodes positioned about the knees, or by a user only placing electrodes into the required pockets. Any known placement of TENS units electrodes for the treatment of pain is intended to be a potential placement of inner pockets for the electrodes in the inventive system.

As an alternative embodiment, not illustrated, the electrical contact may also be achieved by a hole in the inner pocket, which is smaller than the size of the electrode to prevent the electrode falling out of the pocket. However, because this will require more fine coordination to get the electrodes into and out of the pockets, it is not the preferred embodiment. Additionally, the conductive fabric or material takes the place of conductive gel, and therefore the electrode itself must be structured differently for there to not be conductive material forming the wall of the pocket.

As an alternative embodiment, not illustrated, the wires of the TENS unit may be replaced with conductive threads or filaments which are part of the fabric of the garment body or are otherwise permanently attached to it. However, this requires connection of the power source to the threads and connection of the electrodes to the threads. Additionally, this requires the insulation of the wires or filaments from the user, to ensure the electricity gets to the electrodes and therefore to the area in need of pain relief.

As an alternative embodiment, not illustrated, the base garment may be a compression wrap with hook and loop fasteners to permit adjustment of the degree of compression.

Although not illustrated in combination, the use of heat in any form factor of base garment, including a top, is considered to be an embodiment of the invention. Similarly, while only a top and pants have been illustrated, other base garments, such as vests, leg warmers, long gloves, and dresses are disclosed herein. Because different people have problems in different areas, clothing which covers any area approved for the use of a TENS unit device for pain relief is intended to be encompassed by the above disclosure.

While illustrative embodiments of the invention have been shown and described, variations and alternative embodiments will occur to those skilled in the art. Such variations and alternative embodiments can be made without departing from the scope of the invention as defined in the claims.

As used in this specification and the appended claims, the singular forms “a” and “an” indicate a single element, while “the” may refer back to single or plural referents. Unless defined otherwise, all technical and scientific terms used herein have the same meaning as commonly understood by one of ordinary skill in the art to which the disclosure pertains.

The above detailed description of exemplary and preferred embodiments is presented for the purposes of illustration and disclosure in accordance with the requirements of the law. It is intended to be exemplary but not exhaustive, and is not intended to limit the invention to the precise forms described, but only to enable others skilled in the art to understand how the invention may be suited for a particular use of implementation. No limitation is intended by the description of exemplary embodiments which may have included tolerances, feature dimensions, specific operating conditions, engineering specifications, or the like, and which may vary between implementations or with changes to the state of the art, and no such limitation should be implied therefrom. Applicant has made this disclosure with respect to the current state of the art, but also contemplates advancements and that adaptations in the future may take into consideration those advancements in accordance with the then current state of the art. It is intended that the scope of the invention be defined by the Claims as written and equivalents as applicable. Reference to a claim element in the singular is not intended to mean “one and only one” unless explicitly so stated. No claim element herein is intended to be construed under the provisions of 35 U.S.C. 112(f), unless the element is expressly recited using the exact phrase “means for.” and no method or process step herein is to be construed under the provisions of 35 U.S.C. section 112(f) unless the step, or steps, are expressly recited using the exact phrase “step(s) for.”.

1: A system for pain relief, comprising:
a base garment with inner pockets positioned, sized, and made of material suitable to hold TENS unit electrodes in place while in use for pain relief;
a power supply, and
conductive wires.

2: the system of claim 1, wherein the power supply is a standard power supply for a TENS unit.

3: the system of claim 1, wherein the garment is a pair of pants or a shirt.

4: The system of claim 1, wherein garment further comprises a pocket for holding the power supply.

5: The system of claim 1, wherein the inner pockets are configured to be closed using hook and loop fasteners, such that the electrodes can be held securely within the pockets while connected electrically to the power supply.

6: The system of claim 1, wherein the garment is machine-washable after removing the power supply, conductive wires, and TENS unit electrodes.

7. The system of claim 1, further comprising electrically powered heating wires configured to connect to the power supply.

8. The system of claim 4, wherein the pocket for holding the power supply is configured to be closed using hook and loop fasteners, such that the power supply can be held securely within the pocket while connected electrically to the electrodes.

9. The system of claim 4, wherein the pockets for holding the power supply is configured to be closed using hook and loop fasteners, such that the power supply can be held securely within the pocket while connected electrically to the heating wires.
